(19) United States
(12) Patent Application Publication
(10) Pub. No.: US 2025/0325272 A1
(43) Pub. Date: Oct. 23, 2025

(54) SYSTEM FOR OCCLUDING A LEFT ATRIAL APPENDAGE

(71) Applicant: BOSTON SCIENTIFIC SCIMED, INC., MAPLE GROVE, MN (US)

(72) Inventors: Patrick Willoughby, Shoreview, MN (US); Brian Joseph Tischler, Shoreview, MN (US); Emily Vance, Minneapolis, MN (US)

(73) Assignee: BOSTON SCIENTIFIC SCIMED, INC., MAPLE GROVE, MN (US)

(21) Appl. No.: 19/185,820

(22) Filed: Apr. 22, 2025

Related U.S. Application Data
(60) Provisional application No. 63/637,476, filed on Apr. 23, 2024.

(51) Int. Cl. A61B 17/12; A61B 17/00

(52) U.S. Cl. CPC A61B 17/12122; A61B 17/12036; A61B 17/12159; A61B 17/12172; A61B 17/12186; A61B 2017/00867; A61B 2017/1205

(57) ABSTRACT

A system for occluding a patient's left atrial appendage (LAA) includes an expandable frame and one or more expandable elements. The expandable frame is moveable from a collapsed delivery configuration to an expanded deployment configuration in which the expandable frame is adapted to span across at least a portion of the LAA, the expandable frame defining an LAA volume distal of the expandable frame once deployed. The one or more expandable elements are adapted to be deployed behind the expandable frame in order to fill at least part of the LAA volume distal of the expandable frame, each of the one or more expandable elements adapted to expand subsequent to delivery.

### CROSS-REFERENCE TO RELATED APPLICATION

This application is a continuation of U.S. Patent Application Ser. No. 63/637,476, filed April 23, 2024, entitled “SYSTEMS FOR OCCLUDING A LEFT ATRIAL APPENDAGE”, which is incorporated by reference herein in its entirety.

### TECHNICAL FIELD

The disclosure relates generally to medical devices and more particularly to systems for occluding a left atrial appendage.

### BACKGROUND

Medical devices implanted within the heart may include left atrial appendage closure (LAAC) devices, which are intended to close off the left atrial appendage (LAA) in order to reduce the likelihood of thrombi forming in the LAA from escaping the LAA and entering the bloodstream. Thrombi that migrate through the blood vessels may eventually plug a smaller vessel downstream and thereby contribute to stroke or heart attack. Clinical studies have shown that the majority of blood clots in patients with atrial fibrillation originate in the LAA. As a treatment, medical devices have been developed which are deployed to close off the left atrial appendage. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example may be found in a system for occluding a patient's left atrial appendage (LAA). The system includes an expandable frame that is moveable from a collapsed delivery configuration to an expanded deployment configuration in which the expandable frame is adapted to span across at least a portion of the LAA. The expandable frame define an LA A volume distal of the expandable frame once deployed. One or more expandable elements are adapted to be deployed behind the expandable frame in order to fill at least part of the LA A volume distal of the expandable frame. Each of the one or more expandable elements are adapted to expand subsequent to delivery.

Alternatively or additionally, the one or more expandable elements may be adapted to be delivered after the expandable frame is implanted.

Alternatively or additionally, at least some of the one or more expandable elements may include expandable foam blocks adapted to be delivered in a compressed configuration and to expand once delivered into the expanded configuration.

Alternatively or additionally, the system may further include a plurality of expandable foam blocks that a user can select from, where the one or more expandable elements include the expandable foam blocks selected from the plurality of different size expandable foam blocks.

Alternatively or additionally, the plurality of expandable foam blocks may include one or more expandable foam blocks having a first expanded size and one or more expandable foam blocks having a second expanded size different from the first expanded size.

Alternatively or additionally, the expandable foam blocks may include cylindrical foam blocks that are compressed for delivery.

Alternatively or additionally, at least some of the one or more expandable elements may be delivered into the volume distal of the expandable frame as a liquid that subsequently expands within the volume distal of the expandable frame.

Alternatively or additionally, the liquid may include a liquid foam.

Alternatively or additionally, the liquid may include a hydrogel.

Alternatively or additionally, the one or more expandable elements may include a shape memory foam plug shaped to facilitate extending the shape memory foam plug through the expandable frame.

Alternatively or additionally, the shape memory foam may be shaped to include a sharp tip adapted to penetrate through a fabric layer disposed on the expandable frame.

Another example may be found in a kit for occluding a patient's left atrial appendage (LAA). The kit includes an expandable frame that is adapted to be deployed within the LAA in order to at least partially occlude at least part of the LAA and a collection of expandable foam elements. The expandable foam elements may be individually selected and then deployed distal of the expandable frame. Each of the individually selected expandable foam elements are adapted to expand upon deployment.

Alternatively or additionally, the collection of expandable foam elements may include a plurality of expandable foam elements having varying expanded volumes.

Alternatively or additionally, the plurality of expandable foam blocks may include one or more expandable foam blocks having a first expanded size and one or more expandable foam blocks having a second expanded size different from the first expanded size.

Alternatively or additionally, at least some of the collection of expandable foam elements may include cylindrical foam blocks that are compressed for delivery.

Another example may be found in a method of occluding a patient's left atrial appendage (LAA). The method includes deploying an expandable frame to at least partially occlude the LAA, extending a delivery catheter through the expandable frame such that the delivery catheter reaches an LAA volume distal of the deployed expandable frame, and using the delivery catheter to deploy one or more expandable elements within the LAA volume.

Alternatively or additionally, the delivery catheter may be used to deploy one or more expandable foam elements distal of the deployed expandable frame. The one or more expandable foam elements may expand to fill at least part of the LAA volume.

Alternatively or additionally, the method may further include selecting the one or more expandable foam elements for deployment via the delivery catheter from a collection of expandable foam elements that includes expandable foam elements having varying expanded sizes.

Alternatively or additionally, the delivery catheter may be used to deploy an expandable liquid within the LAA volume.

Alternatively or additionally, the method may further include extending a second delivery catheter through the expandable frame at a position spaced from where the delivery catheter was extended and using the second delivery catheter to deliver one or more additional expandable elements into the LAA volume.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1A is a partial cross-sectional view of a left atrial appendage (LAA), showing an expandable frame disposed near the mouth of the LAA;

FIG. 1B is a side view of the LAA of FIG. 1A;

FIGS. 2A, 2B, 2C, 2D and 2E together show an illustrative process for occluding the LAA of FIGS. 1A and 1B;

FIGS. 3A, 3B, 3C and 3D together show an illustrative process for occluding the LAA of FIGS. 1A and 1B;

FIG. 4A is a schematic view of an illustrative delivery catheter usable in the illustrative process of FIGS. 3A, 3B, 3C and 3D;

FIG. 4B is a cross-sectional view of the illustrative delivery catheter of FIG. 4A, taken along the line 4B-4B thereof;

FIG. 5 is a schematic view of a collection of expandable elements that may be selected and used in combination with the illustrative delivery catheter of FIG. 4A as a system or kit;

FIGS. 6A and 6B together show an illustrative occlusive device and method for occluding the LAA; and

FIGS. 7A and 7B together show an illustrative process for occluding a multi-lobe LAA.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale. The detailed description and drawings are intended to illustrate but not limit the present disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the present disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to use the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

A system for occluding a patient's left atrial appendage (LAA) may include several components. The system may include an expandable frame that is moveable from a collapsed delivery configuration to an expanded deployment configuration in which the expandable frame is adapted to span across at least a portion of the LAA, the expandable frame defining an LAA volume distal of the expandable frame once deployed. The system includes one or more expandable elements that are adapted to be deployed behind the expandable frame in order to fill at least part of the LAA volume distal of the expandable frame, each of the one or more expandable elements adapted to expand subsequent to delivery. In some cases, the one or more expandable elements may be adapted to be delivered after the expandable frame is implanted. In some cases, at least some of the one or more expandable elements may include expandable foam blocks adapted to be delivered in a compressed configuration and to expand once delivered into the expanded configuration. The system may, for example, further include a plurality of expandable foam blocks that a user can select from, where the one or more expandable elements include the expandable foam blocks selected from the plurality of different size expandable foam blocks. In some cases, the plurality of expandable foam blocks may include one or more expandable foam blocks having a first expanded size and one or more expandable foam blocks having a second expanded size different from the first expanded size. In some cases, the expandable foam blocks may include cylindrical foam blocks that are compressed for delivery. At least some of the one or more expandable elements may be delivered into the volume distal of the expandable frame as a liquid that subsequently expands within the volume distal of the expandable frame. As an example, the liquid may be a liquid foam. As another example, the liquid may include a hydrogel. In some cases, the one or more expandable elements may include a shape memory foam plug shaped to facilitate extending the shape memory foam plug through the expandable frame. The shape memory foam may be shaped to include a sharp tip adapted to penetrate through a fabric layer disposed on the expandable frame, for example.

A kit for occluding a patient's LAA may include an expandable frame that is adapted to be deployed within the LAA in order to at least partially occlude at least part of the LAA and a collection of expandable foam elements, where expandable foam elements may be individually selected and then deployed distal of the expandable frame. Each of the individually selected expandable foam elements may be adapted to expand upon deployment. In some cases, the collection of expandable foam elements may include a plurality of expandable foam elements having varying expanded volumes. In some cases, the plurality of expandable foam blocks may include one or more expandable foam blocks having a first expanded size and one or more expandable foam blocks having a second expanded size different from the first expanded size. At least some of the collection of expandable foam elements may include cylindrical foam blocks that are compressed for delivery.

A method of occluding a patient's LAA includes deploying an expandable frame to at least partially occlude the LAA and extending a delivery catheter through the expandable frame such that the delivery catheter reaches an LAA volume distal of the deployed expandable frame. The delivery catheter may be used to deploy one or more expandable elements within the LAA volume. In some cases, the delivery catheter may be used to deploy one or more expandable foam elements distal of the deployed expandable frame, where the one or more expandable foam elements expand to fill at least part of the LAA volume. In some cases, the method may further include selecting the one or more expandable foam elements for deployment via the delivery catheter from a collection of expandable foam elements that includes expandable foam elements having varying expanded sizes. In some cases, the delivery catheter may be used to deploy an expandable liquid within the LAA volume. In some cases, the method may further include extending a second delivery catheter through the expandable frame at a position spaced from where the delivery catheter was extended and using the second delivery catheter to deliver one or more additional expandable elements into the LAA volume. In some cases, a single delivery catheter may be used to deliver one or more expandable elements, and then withdrawn and advanced through the expandable frame at a different position to subsequently deliver one or more expandable elements.

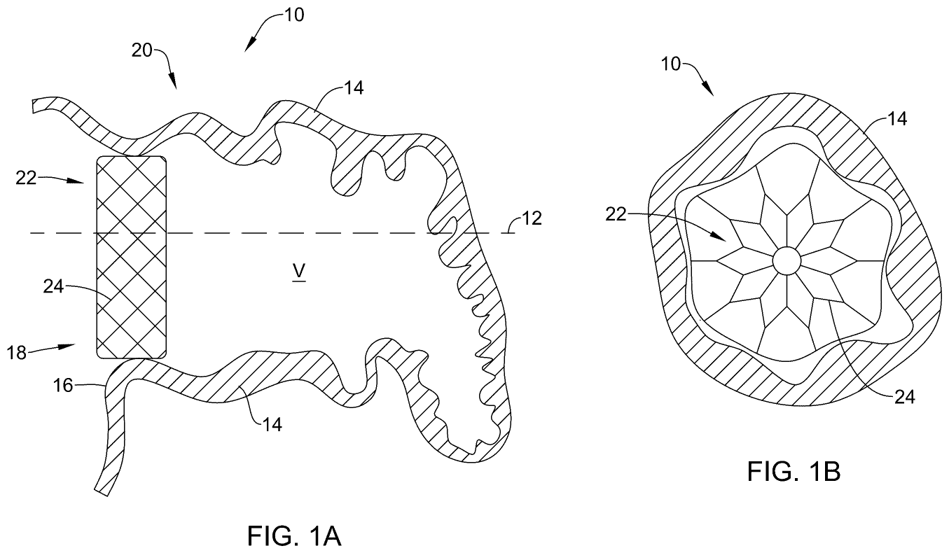

FIG. 1A is a partial cross-sectional view of a left atrial appendage (LAA) 10 and FIG. 1B is an end view of the LAA 10. In some embodiments, the LAA 10 may have a complex geometry and/or irregular surface area. It will be appreciated that the illustrated LAA 10 is merely one of many possible shapes and sizes for the LAA 10, which may vary from patient to patient. Those of skill in the art will also recognize that the medical devices, systems, and/or methods disclosed herein may be adapted for various sizes and shapes of the LAA 10, as necessary. The left atrial appendage 10 may include a generally longitudinal axis 12 arranged along a depth of a main body 20 of the left atrial appendage 10. The main body 20 may include a lateral wall 14 and an ostium 16 forming a proximal mouth 18. In some examples, a lateral extent of the ostium 16 and/or the lateral wall 14 may be smaller or less than a depth of the main body 20 along the longitudinal axis 12, or a depth of the main body 20 may be greater than a lateral extent of the ostium 16 and/or the lateral wall 14. In some examples, the LAA 10 may narrow quickly along the depth of the main body 20 or the left atrial appendage may maintain a generally constant lateral extent along a majority of depth of the main body 20. In some examples, the LAA 10 may include a distalmost region formed or arranged as a tail-like element associated with a distal portion of the main body 20. In some examples, the distalmost region may protrude radially or laterally away from the longitudinal axis 12.

As shown, an expandable frame 22 has been implanted within the LAA 10, near the proximal mouth 18 of the LAA 10. While not shown, the expandable frame 22 may be delivered using a delivery catheter having a central core member. The expandable frame 22 may take any of a variety of different forms. In some instances, the expandable frame 22 may be moveable between a collapsed configuration for delivery and an expanded configuration (as shown) once deployed. The expandable frame 22 may be formed of a shape memory material, such as a shape memory polymer or a shape memory metal. As shown, the expandable frame 22 may include a number of struts 24 that are attached together in forming the expandable frame 22. While not shown, in some cases the expandable frame 22 may include a fabric or other material forming an occlusive cover extending over at least a portion of the metallic or polymeric frame. The WATCHMAN™ implant, which is commercially available from Boston Scientific, is an example of a left atrial appendage closure (LAAC) device having a metallic frame and an occlusive covering extending over at least part of the metallic frame. The expandable frame 22 may be considered as defining a volume V within the LAA 10, distal of the expandable frame 22 and bounded at least in part by the lateral walls 14 of the LAA 10.

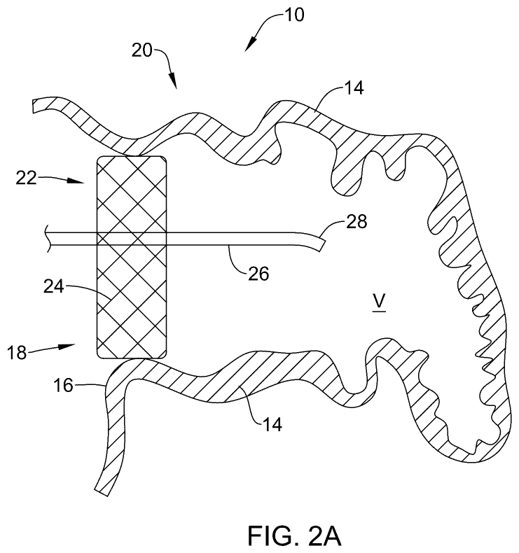

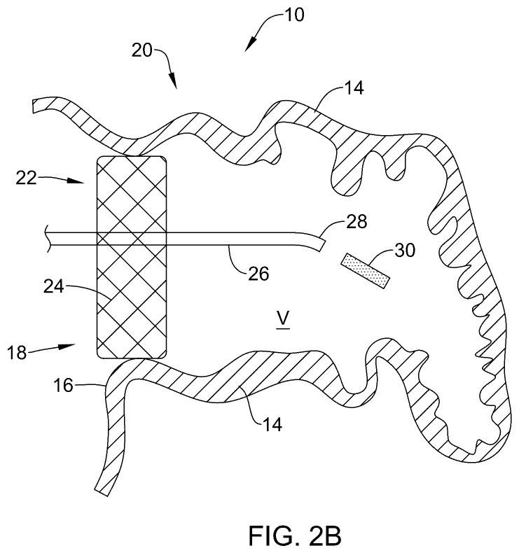

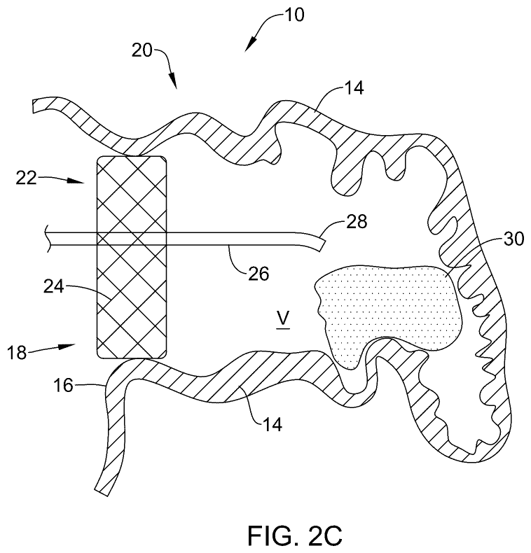

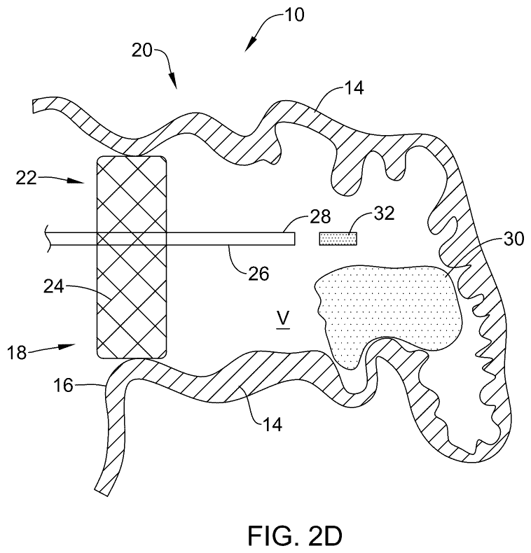

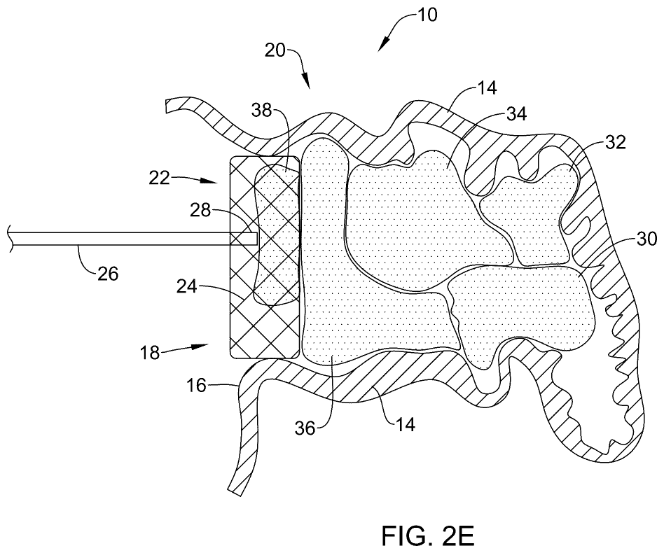

FIGS. 2A through 2E provide a step by step example of an illustrative process of occluding the LAA 10 by first deploying the expandable frame 22 and then using expandable elements to fill at least part of the volume V within the LAA 10. In some cases, the process may also fill at least part of a volume within the expandable frame 22. In FIG. 2A, a delivery catheter 26 is shown as being advanced distally through the expandable frame 22 such that a distal end 28 of the delivery catheter 26 is disposed within the LAA 10, distal of the expandable frame 22. In FIG. 2B, an expandable element 30 has been delivered to the volume V within the LAA 10 by the delivery catheter 26. In some cases, the expandable element 30 may be in liquid form, such as an expandable foam (or precursor thereof) or a hydrogel. The expandable element 30 may be delivered in any suitable quantity. The expandable element 30 may have a volume that is in a range of about 1 milliliter (ml) (about 0.061 cubic inches (in³)) to about 10 ml (about 0.61 in³). As an example, the expandable element 30 may have a volume that is in a range of about 5.5 ml (about 0.34 in³) about 12.5 ml (about 0.76 in³). As another example, the expandable element 30 may have a volume of about 2 milliliters (ml) (about 0.122 in³), although other quantities are contemplated. In cases where the expandable element 30 may be in liquid form, the delivery catheter 26 may include an interior lumen through which the liquid form of the expandable element 30 may be pumped. FIG. 2C shows the expandable element 30 in its expanded form.

In some cases, the expandable element 30 may be a particular amount of a liquid polymeric precursor of an expandable foam that will form an expandable foam once exposed to body temperature blood within the LAA 10. Examples of suitable foam materials are described below. In some cases, the expandable element 30 may be a particular amount of a hydrogel. Examples of possible hydrogels include the Obsidio Conformable Embolic or the SpaceOAR Hydrogel, each of which are commercially available from Boston Scientific. Additional examples include Instylla, Onyx, or cyanoacrylate glues like Histoacryl or A vacryl.

In some cases, a single expandable element 30, even after expansion, may not be sufficient to fill the volume V. In FIG. 2D, the delivery catheter 26 has been withdrawn from its initial position, as shown in FIG. 2A, and extended through the expandable frame 22 at a different location. As shown in FIG. 2D, an expandable element 32 has been delivered into the volume V within the LAA 10. In some cases, the delivery catheter 26 may be moved around multiple times to inject sufficient expandable elements into the volume V. In some cases, a plurality of delivery catheters 26 (only one is shown) may be extended through the expandable frame 22 in order to inject multiple expandable elements into the volume V. In some cases, depending on how quickly the expandable elements solidify or otherwise expand into their expanded configurations, it may be possible to keep the delivery catheter 26 in a particular position, such as extending through a midpoint of the expandable frame 22, while injecting or otherwise delivering a number of expandable elements one immediately after the next.

FIG. 2E shows the LAA 10 filled by a number of expandable elements 30, 32, 34, 36 and 38, which may result in multiple injections without moving the delivery catheter 26, or by extending the delivery catheter 26 through the expandable frame 22 in multiple positions relative to the expandable frame 22. In some cases, the expandable elements 30, 32, 34, 36 and 38, and possibly additional expandable elements, may fill all or substantially all of the volume V within the LAA 10, distal of the expandable frame 22. Over time, any blood within the LAA 10 may clot within open pores within the expandable elements 30, 32, 34, 36 and 38, and possibly additional expandable elements, and convert into thrombus and eventually into native tissue. As a result, the volume V within the LAA 10 will eventually be filled with native tissue, meaning that there is no or essentially no space within the LAA 10 for any blood. No blood means no stagnant blood. In some cases, the expandable elements 30, 32, 34, 36 and 38, and possibly additional expandable elements, may degrade over time, leaving only native tissue behind.

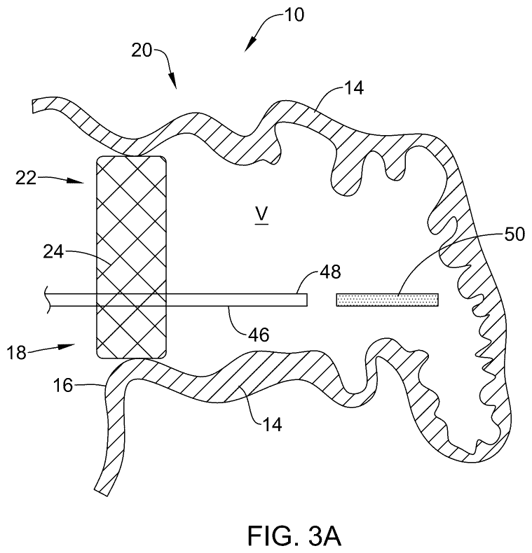

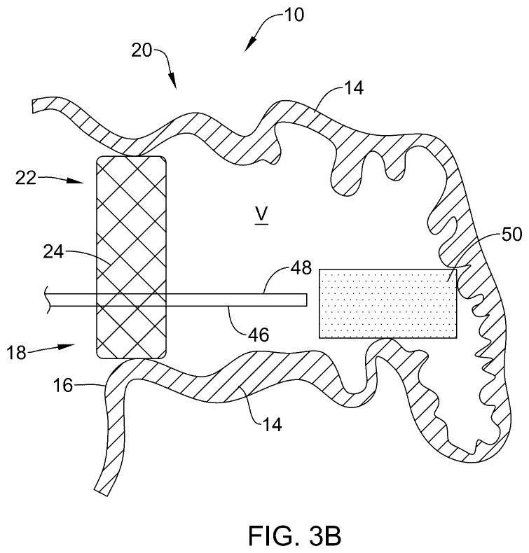

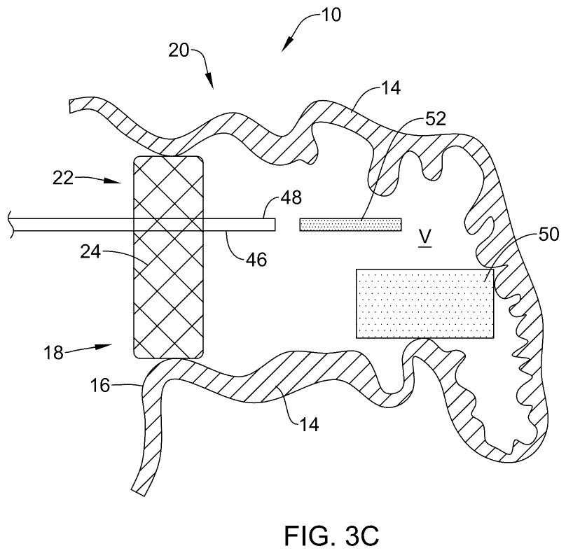

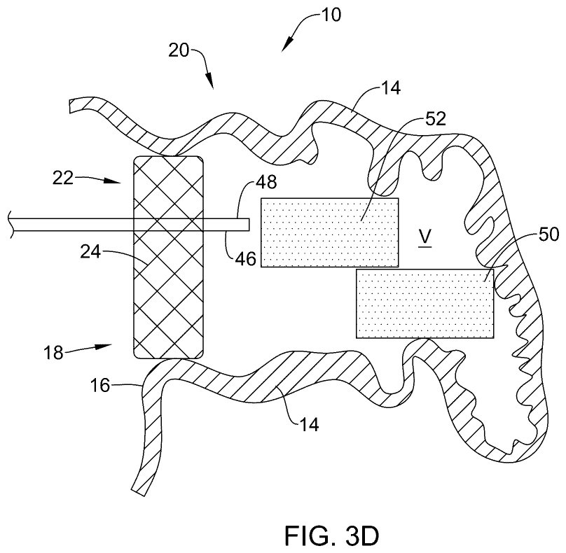

FIGS. 3A through 3D provide a step by step example of an illustrative method of occluding the LAA 10 by first deploying the expandable frame 22 and then using expandable elements to fill at least part of the volume V within the LAA 10. In some cases, the process may also fill at least part of a volume within the expandable frame 22. While FIGS. 2A through 2E show the use of expandable elements that are deployed within the volume V within the LAA 10 in a liquid form, FIGS. 3A through 3D show the use of expandable elements that are deployed within the volume V within the LAA 10 as expandable foam elements. For example, in some cases, the expandable elements shown in FIGS. 3A through 3D may be delivered as expandable foam cylinders that are compressed or crimped into a reduced diameter delivery configuration that will expand into their expanded configurations (sometimes still cylindrical) once released from within a delivery catheter and/or once exposed to body temperature blood within the LAA 10.

In FIG. 3A, a delivery catheter 46 is shown as being advanced distally through the expandable frame 22 such that a distal end 48 of the delivery catheter 46 is disposed within the LAA 10, distal of the expandable frame 22. An expandable element 50 has been delivered to the volume V within the LAA 10 by the delivery catheter 46. As shown, the expandable element 50 is an expandable foam element and is delivered to the volume V within the LAA 10 in its compressed or crimped configuration. As shown in FIG. 3B, the expandable element 50 will expand into its expanded configuration.

In some cases, a single expandable element 50, even after expansion, may not be sufficient to fill the volume V. In FIG. 3C, the delivery catheter 46 has been withdrawn from its initial position, as shown in FIG. 3A, and extended through the expandable frame 22 at a different location. As shown in FIG. 3C, an expandable element 52 has been delivered into the volume V within the LAA 10. In some cases, the delivery catheter 46 may be moved around multiple times to inject sufficient expandable elements into the volume V. The expandable element 52 will then expand, as shown in FIG. 3D. In some cases, additional expandable elements may be injected into the volume V to help fill up the volume V, even though only two expandable elements 50 and 52 are shown in FIGS. 3A through 3D. In some cases, when the expandable frame 22 may be delivered using a delivery catheter having a central core member (not shown), the expandable elements may be advanced through a lumen extending through the central core member of the delivery catheter, instead of or even in addition to advancing the expandable elements through the delivery catheter 46.

In some cases, the expandable elements 50 and 52, and possibly additional expandable elements, may fill all or substantially all of the volume V within the LAA 10, distal of the expandable frame 22. In some cases, the expandable elements 50 and 52 may fill at least some of a volume within the expandable frame 22. Over time, any blood within the LAA 10 may clot within open pores within the expandable elements 50 and 52, and possibly additional expandable elements, and convert into thrombus and eventually into native tissue. As a result, the volume V within the LAA 10 will eventually be filled with native tissue, meaning that there is no or essentially no space within the LAA 10 for any blood. No blood means no stagnant blood. In some cases, the expandable elements 50 and 52, and possibly additional expandable elements, may degrade over time, leaving only native tissue behind.

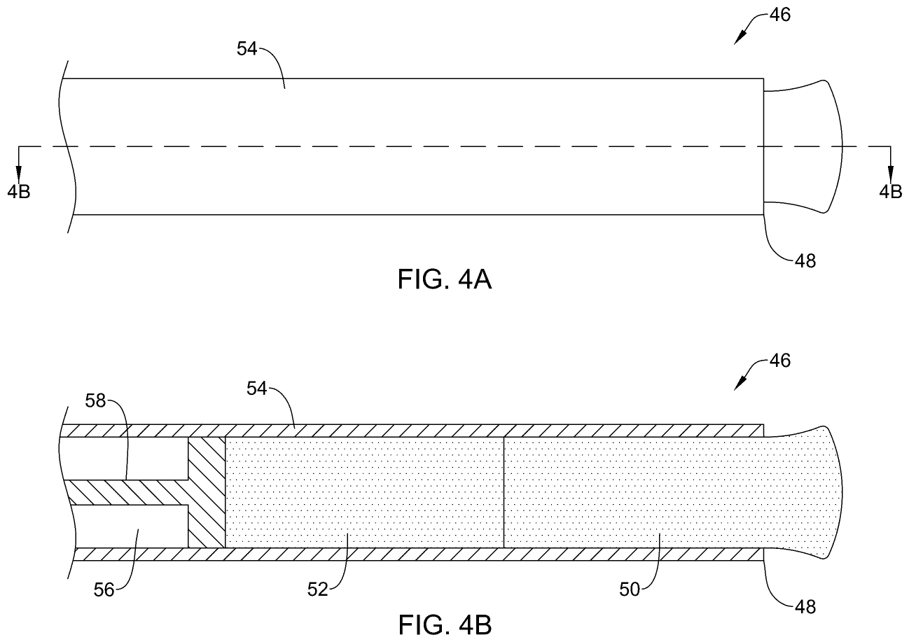

FIG. 4A is a schematic view of the delivery catheter 46, and FIG. 4B is a cross-sectional view thereof, taken along the line 4B-4B of FIG. 4A. The delivery catheter 46 includes an elongate shaft 54 that defines a lumen 56 extending within the elongate shaft 54. As shown, the expandable element 50 and the expandable element 52 are each shown disposed within the lumen 56, and are being pushed distally by a plunger 58. Pushing the plunger 58 a distance equal to a length of the expandable element 50 will cause the expandable element 50 to pass through the distal end 48 of the delivery catheter 46, and to be delivered into the volume V of the LAA 10, and will result in the expandable element 52 to be positioned where the expandable element 50 is currently shown, and thus the expandable element 52 will be positioned to be subsequently delivered by advancing the plunger 58 a distance equal to a length of the expandable element 52. In cases where the expandable elements are delivered in liquid form (such as with the delivery device 26), the plunger 58 may be replaced with a valve that is adapted to open and close in order to release a volume of liquid expandable element.

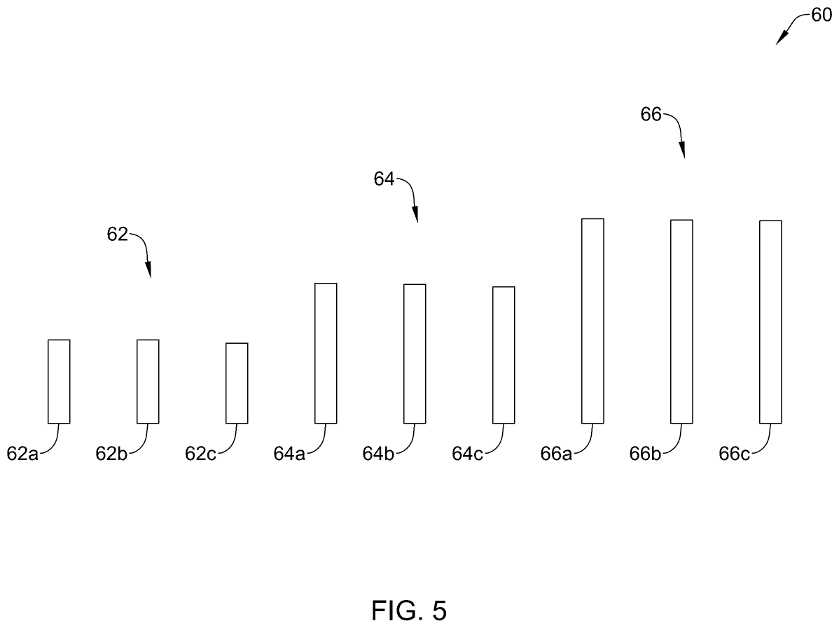

FIG. 5 is a schematic view of a collection of expandable elements 60 that may be selected and used in combination with the illustrative delivery catheter 46 as a system or kit. In some cases, the collection of expandable elements 60 may include several expandable elements 62, individually labeled as 62a, 62b and 62c, having a first compressed or crimped size. The collection of expandable elements 60 may include several expandable elements 64, individually labeled as 64a, 64b and 64c, having a second compressed or crimped size. The collection of expandable elements 60 may include several expandable elements 66, individually labeled as 66a, 66b and 66c, having a third compressed or crimped size. In some cases, at least some of the expandable elements 62, at least some of the expandable elements 64 and/or at least some of the expandable elements 66 may have cylindrical profiles, at least when compressed.

While a total of three expandable elements 62, a total of three expandable elements 64 and a total of three expandable elements 66 are shown, this is merely illustrative as the collection of expandable elements 60 may include any number of expandable elements 62, any number of expandable elements 64 and any number of expandable elements 66. Depending on the interior volume of the LAA 10 being filled, a user may select particular combinations of the expandable elements 62, the expandable elements 64 and/or the expandable elements 66 to fill up the volume V of the LAA 10 distal to the expandable frame 22. In some cases, the expandable elements 64 and/or the expandable elements 66 may fill up at least part of a volume within the expandable frame 22.

As shown, each of the expandable elements 62 have a length, each of the expandable elements 64 have a length that is greater than the length of each of the expandable elements 62, and each of the expandable elements 66 have a length that is greater than the length of each of the expandable elements 64. As an example, the expandable elements 62, 64, and 66 may have a length that ranges from about 10 millimeters (mm) (about 0.39 inches) to about 30 mm (about 1.18 inches) when expanded. In some cases, the expandable elements 62, 64 and 66 may change only in diameter when expanding, and may not change in length. Each of the expandable elements 62, 64 and 66 may have a compressed diameter that ranges from about 0.5 mm (about 0.02 inches) to about 4 mm (about 0.16 inches), changing to an expanded diameter that ranges from about 10 mm (about 0.39 inches) to about 50 mm (about 1.97 inches). For particular examples, one of the expandable elements 62 may have compressed dimensions of about 1 mm (about 0.039 inches) by about 15 mm (about 0.59 inches) and expanded dimensions of about 12 mm (about 0.47 inches) by about 15 mm (about 0.59 inches), or compressed dimensions of about 0.5 mm (about 0.02 inches) by about 12 mm (about 0.47 inches) and expanded dimensions of about 6 mm (about 0.24 inches) by about 15 mm (about 0.59 inches), or compressed dimensions of about 3.2 mm (about 0.13 inches) by about 15 mm (0.59 inches) and expanded dimensions of about 32 mm (about 1.26 inches) by about 15 mm (about 0.59 inches).

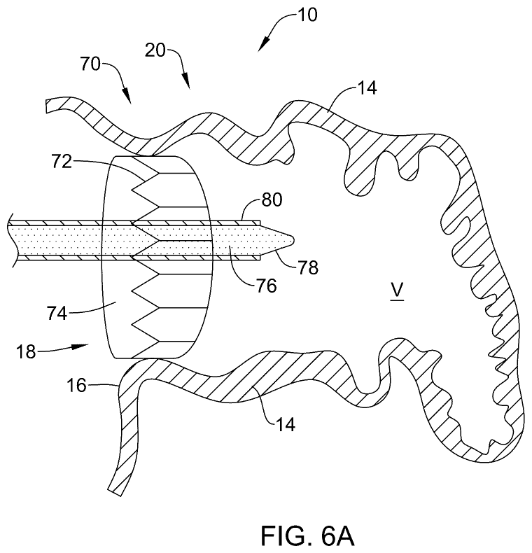

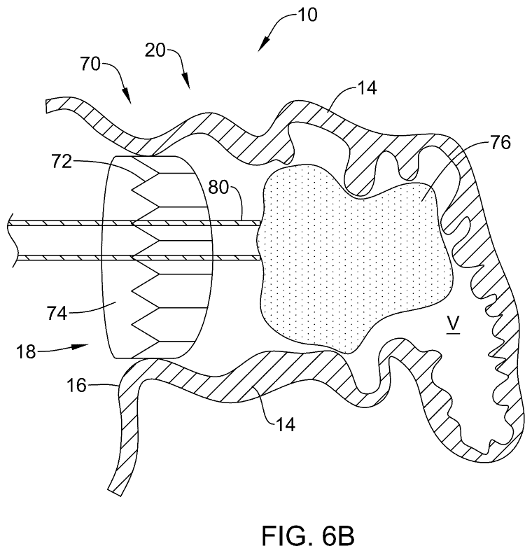

FIGS. 6A and 6B together show an illustrative occlusive device 70 for occluding the LAA 10 as well as a method of using the occlusive device 70. As shown in FIG. 6A, the occlusive device 70 includes an expandable frame 72 that includes an occlusive covering 74 extending over at least a proximal portion of the expandable frame 72. The expandable frame 72 and the occlusive covering 74, in combination, may be considered as being the aforementioned WATCHMAN™ implant. The occlusive device 70 includes a shape memory foam plug 76 that is crimped and formed to have a sharp tip 78. The shape memory foam plug 76 may be loaded into a delivery sheath 80 that may be placed in direct contact with the occlusive covering 74. The shape memory foam plug 76 may be advanced distally relative to the delivery sheath 80 such that the sharp tip 78 contacts the occlusive covering 74. The sharp tip 78 may penetrate through the occlusive covering 74 such that the shape memory foam plug 76 is advanced through the occlusive covering 74 and through the expandable frame 72 in order to reach the volume V distal of the expandable frame 72. In some cases, the shape memory foam plug 76 may fill at least a portion of the volume within the expandable frame 72. As seen for example in FIG. 6B, the shape memory foam plug 76 may be allowed to expand, thereby filling at least a portion of the volume V. While not shown, the occlusive device 70 may include a plunger similar to the plunger 58, or perhaps a push wire, to urge the shape memory foam plug 76 distally through and out of the delivery sheath 80.

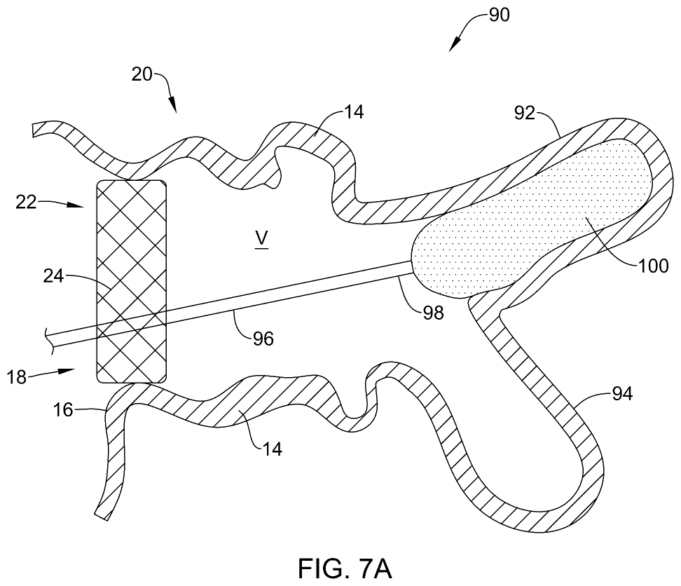

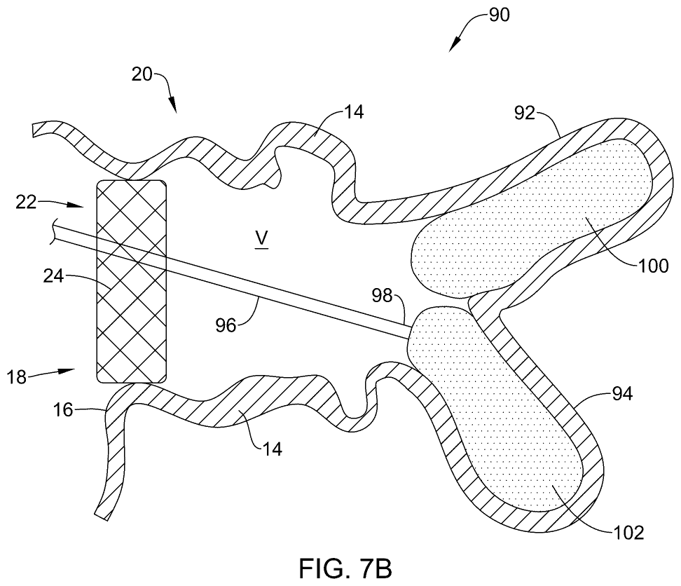

FIGS. 7A and 7B together show a process for occluding an LAA 90 that includes some of the features of the LAA 10, including the main body 20 that includes the lateral wall 14 and the ostium 16 forming the proximal mouth 18. In some cases, the LAA 90 may include a first distal lobe 92 and a second distal lobe 94. It will be appreciated that in order to occlude the LAA 90, and fill the volume V with expandable material, it may be helpful to first fill the first distal lobe 92 and the second distal lobe 94 before filling the more proximal portions of the volume V. In FIGS. 7A and 7B, the expandable frame 22 has been deployed within the LAA 90, spanning across the proximal mouth 18. As seen in FIG. 7A, a delivery catheter 96 having a distal end 98 has been advanced through the expandable frame 22 with its distal end 98 positioned next to the first distal lobe 92. An expandable element 100 has been delivered through the delivery catheter 98 and into the first distal lobe 92 and has expanded. The expandable element 100 may be delivered in liquid form, or as a compressed foam element. As seen in FIG. 7B, the delivery catheter 96 has been repositioned with its distal end 98 positioned next to the second distal lobe 94. An expandable element 102 has been delivered through the delivery catheter 98 and into the second distal lobe 94 and has expanded. The expandable element 102 may be delivered in liquid form, or as a compressed foam element. Now that the first distal lobe 92 and the second distal lobe 94 have been filled, the rest of the volume V may now be filled. This may also include filling at least some of a volume within the expandable frame 22.

The expandable foam may include any suitable material, such as a suitable polymeric material, that is capable of transitioning from an initial configuration to an expanded configuration upon being subjected to a specific temperature or temperature range and/or exposure to moisture, and provide a suitable density in the expanded configuration for use inside of the left atrial appendage to provide an occlusive benefit without negatively impacting surrounding anatomy. In some instances, the expandable foam may be a shape memory foam. Suitable transition temperatures may be, for example, at or below about 37° C. (98.6° F.), which allows the shape memory foam to assume an initial configuration prior to and during delivery through a delivery catheter or other delivery device, and an expanded configuration for occlusion after delivery and release within the left atrial appendage, allowing the shape memory foam to be exposed to body temperature blood within the left atrial appendage. A suitable density of the shape memory foam in the expanded configuration is a density that allows the expanded configuration to be pliable and compliant and substantially conform to the left atrial appendage anatomy to create a seal to protect against the formation and escape of blood clots while having sufficient radial force to seal the left atrial appendage but not damage or impact surrounding anatomy. In some instances, the density of the shape memory foam in the expanded configuration will be from about 10 kg/m³ (about 0.62 lb/ft³) to about 1000 kg/m³ (about 62.31 lb/ft³), including from about 10 kg/m³ to about 500 kg/m³ (about 31.2 lb/ft³) including from about 10 kg/m³ to about 200 kg/m³ (about 12.5 lb/ft³), including from about 20 kg/m³ to about 100 kg/m³ (about 6.2 lb/ft³).

Generally, the material for constructing the shape memory foam is a polymeric material that is both biocompatible and substantially biostable. In some instances, biocompatibility will include meeting or surpassing the requirements of established standards for implant materials defined in ISO 10993 and USP Class VI. Substantially biostable materials include those materials that do not resorb over the intended lifetime of the medical device (such as five years, or ten years, or longer), as well as those materials that resorb slowly such that void volume is replaced by a stable tissue-like material over a period of a few months to a year.

In some instances, the shape memory foam may include a natural and/or synthetic material. Suitable natural materials may include, for example, extracellular matrix (ECM) biopolymers such as collagen, fibronectin, hyaluronic acid and elastin, non-ECM biomaterials such as cross-linked albumin, fibrin, and inorganic bioceramics such as hydroxyapatite and tricalcium phosphate. Suitable synthetic materials may include, for example, biostable polymers such as saturated and unsaturated polyolefins including polyethylene, polyacrylics, polyacrylates, polymethacrylates, polyamides, polyimides, polyurethanes, polyureas, polyvinyl aromatics such as polystyrene, polyisobutylene copolymers and isobutylene-styrene block copolymers such as styrene-isobutylene-styrene tert-block copolymers (SIBS), polyvinylpyrolidone, polyvinyl alcohols, copolymers of vinyl monomers such as ethylene vinyl acetate (EVA), polyvinyl ethers, polyesters including polyethylene terephthalate, polyacrylamides, polyethers such as polyethylene glycol, polytetrahydrofuran and polyether sulfone, polycarbonates, silicones such as siloxane polymers, and fluoropolymers such as polyvinylidene fluoride, and mixtures and copolymers of the above.

In some instances, the shape memory foam may include a bioresorbable material such that resorption results in the formation of a biostable tissue matrix. Synthetic bioresorbable polymers may, for example, be selected from the following: (a) polyester homopolymers and copolymers such as polyglycolide (PGA; polyglycolic acid), polylactide (PLA; polylactic acid) including poly-L-lactide, poly-D-lactide and poly-D,L-lactide, poly(beta-hydroxybutyrate), polygluconate including poly-D-gluconate, poly-L-gluconate, poly-D,L-gluconate, poly(epsilon-caprolactone), poly(delta-valerolactone), poly(p-dioxanone), poly(lactide-co-glycolide) (PLGA), poly(lactide-codelta-valerolactone), poly(lactide-co-epsilon-caprolactone), poly(lactide-co-beta-malic acid), poly(beta-hydroxybutyrate-co-beta hydroxyvalerate), poly[1,3bis(p-carboxyphenoxy)propane-co-sebacic acid], and poly(sebacic acid-co-fumaric acid); (b) polycarbonate homopolymers and copolymers such as poly(trimethylene carbonate), poly(lactide-co-trimethylene carbonate) and poly(glycolide-co-trimethylene carbonate); (c) poly(ortho ester homopolymers and copolymers such as those synthesized by copolymerzation of various diketene acetals and diols; (d) polyanhydride homopolymers and copolymers such as poly(adipic anhydride), poly(suberic anhydride), poly (sebacic anhydride), poly(dodecanedioic anhydride), poly(maleic anhydride), poly[1,3-bis-(p-carboxyphenoxy)methane anhydride], and poly[alpha,omega-bis(p-carboxyphenoxy)alkane anhydride] such as poly[1,3-bis(p-carboxyphenoxy)propane anhydride] and poly[1,3-bis(p-carboxyphenoxy)hexane anhydride]; (e) polyphosphazenes such as aminated and alkoxy substituted polyphosphazenes; and (f) amino-acid-based polymers including tyrosine-based polymers such as tyrosine-based polyacrylates (e.g., copolymers of a diphenol and a diacid linked by ester bonds, with diphenols selected, for example, from ethyl, butyl, hexyl, octyl, and benzyl esters of desaminotyrosyl-tyrosine and diacids selected, for example, from succinic, glutaric, adipic, suberic, and sebacic acid), tyrosine-based polycarbonates (e.g., copolymers formed by the condensation polymerization of phosgene and a diphenol selected, for example, from ethyl, butyl, hexyl, octyl, and benzyl esters of desaminotyrosyl-tyrosine, tyrosine-based iminocarbonates, and tyrosine-, leucine- and lysine-based polyester-amides; specific examples of tyrosine-based polymers further include polymers that are comprised of a combination of desaminotyrosyl tyrosine hexyl ester, desaminotyrosyl tyrosine, and various di-acids, for example, succinic acid and adipic acid. Suitable materials include cross-linked polycarbonates and crosslinked polyethylene glycols.

In some instances, the shape memory foam may include thermoset polyurethanes that include oxidatively susceptible linkages in the soft segment, including but not limited to tertiary amines and polyethers. The shape memory foam may optionally include hydrolytically degradable soft segment components such as polycaprolactone, esters, and others.

A shape memory foam may have a thermal transition point (transition temperature) below which residual stress is maintained without a loading constraint. The thermal activation (which causes the shape memory) may be achieved with the desired material going through a semi-crystalline melt point or glass transition temperature between the first configuration and the expanded configuration. Several suitable thermal activation processes are known in the art and useful herein. In an example, the temperature activated memory shape foam may be formed for use as a medical device by first shaping a shape memory foam formed from a suitable material into its final expanded configuration; that is, the configuration that the shape memory shape foam will achieve once inserted into the left atrial appendage to provide the desired occlusive benefit. In this expanded configuration, the shape memory foam may generally have a diameter that will range from about 10 millimeters (about 0.39 inches) to about 50 millimeters (about 1.97 inches) and a length that will range from about 1 centimeter (about 0.39 inches) to about 5 centimeters (about 1.97 inches), although other diameters and lengths are within the scope of the present disclosure. Once this has been done, the foam may be heated above the transition temperature of the material; that is, the temperature at which a desired expansion will occur. Once the desired transition temperature has been achieved, the shape memory foam is held at a constant temperature and is re-shaped into an initial (unexpanded) configuration. This re-shaping is suitably done in a properly sized molding element and may be any suitable shape.

After insertion into the molding element, the temperature is reduced to a temperature below the transition temperature to set the new shape; for example, the temperature may be reduced to room temperature to set the new shape. Once this has been completed, the shape memory foam will remain in its first configuration until it is subjected to a temperature at or above the transition temperature, at which time it will expand into its expanded, or remembered, configuration. In some instances, exposure to water within the blood changes the glass transition temperature of the foam. As an example, the shape memory foam may have a dry T_{g} (glass transition temperature) that is above body temperature, and may have a wet T_{g}, after exposure to water, that is lower than body temperature.

In some cases, the shape memory polymers may include non-foamed versions of the polymers described herein with respect to making the expandable foams such as shape memory foams. Example of bio-compatible shape memory polymers include polymers made from poly(ε-caprolactone) (PCL), polyurethane (PU), poly (D, L-lactide) (PDLLA), PVA, ethylene vinyl acetate copolymer, (EVA) polymer blend, polymer composites, crosslinked polymers and supramolecular networks, among others. In some instances, shape memory polymers that may be used in creating the anchors described herein may include polyurethane, for example.

The materials that can be used for the devices described herein may include those commonly associated with medical devices. The devices described herein, or components thereof, may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHY NOX®, and the like); platinum enriched stainless steel; titanium; combinations thereof; and the like; or any other suitable material.

In at least some embodiments, the devices described herein, or components thereof, may also be doped with, made of, or otherwise include a radiopaque material. Radiopaque materials are understood to be materials capable of producing a relatively bright image on a fluoroscopy screen or another imaging technique during a medical procedure. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of guidewire 10 to achieve the same result.

In some embodiments, a degree of Magnetic Resonance Imaging (MRI) compatibility is imparted into the devices described herein, or components thereof. For example, the devices described herein, or components thereof, may be made of a material that does not substantially distort the image and create substantial artifacts (e.g., gaps in the image). Certain ferromagnetic materials, for example, may not be suitable because they may create artifacts in an MRI image. The devices described herein, or components thereof, may also be made from a material that the MRI machine can image. Some materials that exhibit these characteristics include, for example, tungsten, cobalt-chromium-molybdenum alloys (e.g., UNS: R30003 such as ELGILOY®, PHY NOX®, and the like), nickel-cobalt-chromium-molybdenum alloys (e.g., UNS: R30035 such as MP35-N® and the like), nitinol, and the like, and others.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PV dC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the exterior surface of the devices described herein may be sandblasted, beadblasted, sodium bicarbonate-blasted, electropolished, etc. In these as well as in some other embodiments, a coating, for example a lubricious, a hydrophilic, a protective, or other type of coating may be applied. Alternatively, a sheath may include a lubricious, hydrophilic, protective, or other type of coating. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

Portions of the devices described herein may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present disclosure.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A system for occluding a patient's left atrial appendage (LAA), the system comprising:
an expandable frame moveable from a collapsed delivery configuration to an expanded deployment configuration in which the expandable frame is adapted to span across at least a portion of the LAA, the expandable frame defining an LAA volume distal of the expandable frame once deployed; and
one or more expandable elements adapted to be deployed behind the expandable frame in order to fill at least part of the LAA volume distal of the expandable frame, each of the one or more expandable elements adapted to expand subsequent to delivery.

2. The system of claim 1, wherein the one or more expandable elements are adapted to be delivered after the expandable frame is implanted.

3. The system of claim 1, wherein at least some of the one or more expandable elements comprise expandable foam blocks adapted to be delivered in a compressed configuration and to expand once delivered into the expanded configuration.

4. The system of claim 3, further comprising a plurality of expandable foam blocks that a user can select from, where the one or more expandable elements comprise the expandable foam blocks selected from the plurality of different size expandable foam blocks.

5. The system of claim 4, wherein the plurality of expandable foam blocks include one or more expandable foam blocks having a first expanded size and one or more expandable foam blocks having a second expanded size different from the first expanded size.

6. The system of claim 3, wherein the expandable foam blocks comprise cylindrical foam blocks that are compressed for delivery.

7. The system of claim 1, wherein at least some of the one or more expandable elements are delivered into the volume distal of the expandable frame as a liquid that subsequently expands within the volume distal of the expandable frame.

8. The system of claim 7, wherein the liquid comprises a liquid foam.

9. The system of claim 7, wherein the liquid comprises a hydrogel.

10. The system of claim 1, wherein the one or more expandable elements comprise a shape memory foam plug shaped to facilitate extending the shape memory foam plug through the expandable frame.

11. The system of claim 10, wherein the shape memory foam is shaped to include a sharp tip adapted to penetrate through a fabric layer disposed on the expandable frame.

12. A kit for occluding a patient's left atrial appendage (LAA), the kit comprising:
an expandable frame that is adapted to be deployed within the LAA in order to at least partially occlude at least part of the LAA; and
a collection of expandable foam elements, where expandable foam elements may be individually selected and then deployed distal of the expandable frame;
wherein each of the individually selected expandable foam elements are adapted to expand upon deployment.

13. The kit of claim 12, wherein the collection of expandable foam elements includes a plurality of expandable foam elements having varying expanded volumes.

14. The kit of claim 13, wherein the plurality of expandable foam blocks include one or more expandable foam blocks having a first expanded size and one or more expandable foam blocks having a second expanded size different from the first expanded size.

15. The kit of claim 12, wherein at least some of the collection of expandable foam elements include cylindrical foam blocks that are compressed for delivery.

16. A method of occluding a patient's left atrial appendage (LAA), the method comprising:
deploying an expandable frame to at least partially occlude the LAA;
extending a delivery catheter through the expandable frame such that the delivery catheter reaches an LAA volume distal of the deployed expandable frame; and
using the delivery catheter to deploy one or more expandable elements within the LAA volume.

17. The method of claim 16, wherein the delivery catheter is used to deploy one or more expandable foam elements distal of the deployed expandable frame, where the one or more expandable foam elements expand to fill at least part of the LAA volume.

18. The method of claim 17, further comprising selecting the one or more expandable foam elements for deployment via the delivery catheter from a collection of expandable foam elements that includes expandable foam elements having varying expanded sizes.

19. The method of claim 16, wherein the delivery catheter is used to deploy an expandable liquid within the LAA volume.

20. The method of claim 16, further comprising:
extending a second delivery catheter through the expandable frame at a position spaced from where the delivery catheter was extended; and
using the second delivery catheter to deliver one or more additional expandable elements into the LAA volume.
